(19) United States
(12) Patent Application Publication
(10) Pub. No.: US 2016/0113858 A1
(43) Pub. Date: Apr. 28, 2016

(54) HEAT ACTIVATED ANTIPERSPIRANT

(71) Applicant: The Dial Corporation, Scottsdale, AZ (US)

(72) Inventor: Catherine Schmit, Glendale, AZ (US)

(21) Appl. No.: 14/521,650

(22) Filed: Oct. 23, 2014

(51) Int. Cl. A61K 8/891; A61K 8/86; A61Q 15/00

(52) U.S. Cl. CPC A61K 8/891; A61Q 15/00; A61K 8/86

(57) ABSTRACT

Methods and apparatus are provided for a heat activated antiperspirant. The heat activated antiperspirant composition includes at least one delayed release component to attract the antiperspirant active ingredient. The at least one delayed release component is a solid up to about 33 degrees Celsius (° C.) and has a melting point of about 33 to 41° C. to release the antiperspirant active ingredient to reduce perspiration.

### FIELD OF THE INVENTION

The present invention generally relates to a heat activated antiperspirant compositions and products and more particularly relates to a heat activated antiperspirant compositions and products that includes at least one delayed release component that is a solid at temperatures up to about 33 degrees Celsius (° C.) and has a melting point of about 34° C. to 41° C. to release an antiperspirant active ingredient to reduce perspiration.

### BACKGROUND OF THE INVENTION

Antiperspirants are applied to the skin of a user, for example the underarm. The antiperspirant is used to reduce perspiration of the user by affecting the user's sweat glands. For example, sweat glands, such as the eccrine glands, when stimulated, produce perspiration to cool down a user's body. Perspiration, such as eccrine sweat, is produced as a response to heat exposure, physical exertion, stress, or nervousness. When the sweat glands are stimulated secretory cells in the gland secrete a fluid that travels from a coiled portion of the sweat glands up through a straight duct and onto the surface of the user's skin. Further, antiperspirants have a water soluble zirconium and/or aluminum-based compound as their antiperspirant active ingredient. In one example, the water soluble zirconium and/or aluminum-based compound may be an acidic metal salt. The acidic metal salt in the antiperspirant active ingredient diffuses into the eccrine gland ducts at an opening of the epidermis. The slow neutralization of the acidic metal salt during diffusion into the ducts forms a gelatinous insoluble polymeric aluminum hydroxide-protein plug. As a result, the antiperspirant active ingredient may partially plug the sweat glands to reduce perspiration.

Accordingly, it is desirable to have an antiperspirant that reduces a user's perspiration. In addition, it is desirable to have an antiperspirant that releases the antiperspirant active ingredient over a longer period of time. Furthermore, other desirable features and characteristics of the present invention will become apparent from the subsequent detailed description of the invention and the appended claims, taken in conjunction with the background of the invention.

### BRIEF SUMMARY OF THE INVENTION

A heat activated antiperspirant composition can include an antiperspirant active ingredient, and at least one delayed release component. The at least one delayed release component can be a solid at temperatures up to about 33 degrees Celsius (° C.) and has a melting point of about 33 to 41° C. to release the antiperspirant active ingredient to reduce perspiration.

A heat activated antiperspirant product can include a container and a heat activated antiperspirant composition disposed within the container. The heat activated antiperspirant composition can include an antiperspirant active ingredient and at least one delayed release component. The at least one water soluble cosmetic ingredient can be a solid at temperatures up to about 33° C. and has a melting point of about 33 to 41° C. to release the antiperspirant active ingredient to reduce perspiration.

A heat activated antiperspirant composition can include an antiperspirant active ingredient, at least one delayed release component, and a carrier. The at least one delayed release component is a solid up to about 33° C. and has a melting point of about 33° C. to about 41° C. to release the antiperspirant active ingredient to create vacancies in the carrier. The vacancies allow perspiration to access and dissolve the antiperspirant active ingredient. The at least one delayed release component forms about 0.02 wt % to about 5 wt % of the heat activated antiperspirant composition.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

Antiperspirants are used to reduce perspiration of a user by affecting the user's sweat glands. Antiperspirants are made with several ingredients, which may include a fatty alcohol, a liquid emollient, a volatile silicone, and an antiperspirant active ingredient to reduce perspiration of the user. Further, antiperspirants may have a zirconium and/or an aluminum-based acidic metal salt compound as their antiperspirant active ingredient. The acidic metal salt in the antiperspirant active ingredient diffuses into the eccrine gland ducts at an opening of the epidermis. The slow neutralization of the acidic metal salt during diffusion into the ducts forms a gelatinous insoluble polymeric aluminum hydroxide-protein plug. As a result, the antiperspirant active ingredient may partially plug the sweat glands to reduce perspiration.

The food and drug administration (FDA) sets standards for antiperspirants. For example, the FDA specifies that antiperspirants are to reduce perspiration of a user by at least twenty percent. Further, the FDA specifies that antiperspirants are to reduce perspiration by at least twenty percent in fifty percent of its users.

The heat activated antiperspirant composition includes an antiperspirant active ingredient. As used in the present specification and in the appended claims, an antiperspirant active ingredient refers to any compound used to prevent, reduce, eliminate, or otherwise combat perspiration. In one example, the antiperspirant active ingredient may be an aluminum-based compound such as aluminum or aluminum zirconium. For example, the antiperspirant active ingredient may be aluminum chlorohydrate, aluminum chlorohydrate solution, aluminum sesquichlorohydrate, aluminum chlorohydrex PG, aluminum zirconium trichlorohydrate, aluminum zirconium trichlorohydrex GLY, aluminum zirconium tetrachlorohydrate, aluminum zirconium tetrachlorohydrex GLY, aluminum zirconium tetrachlorohydrex GLY solution, aluminum zirconium pentachlorohydrate, aluminum zirconium octachlorohydrate, aluminum zirconium octachlorohydrex GLY, other zirconium and/or aluminum-based compounds, or combinations thereof. The heat activated antiperspirant composition may be applied to the skin of a user, for example an underarm of the user, to reduce perspiration of the user. As mentioned above, the acidic metal salt in the antiperspirant active ingredient diffuses into the eccrine gland ducts at an opening of the epidermis. The slow neutralization of the acidic metal salt during diffusion into the ducts forms a gelatinous insoluble polymeric aluminum hydroxide-protein plug. As a result, the antiperspirant active ingredient may partially plug the sweat glands to reduce perspiration. More information about the antiperspirants active ingredient will be described in later parts of this specification.

The heat activated antiperspirant composition includes at least one delayed release component. The delayed release component can be a water soluble cosmetic ingredient. The delayed release component can act to attract, co-mingle, partition, or otherwise interact with the antiperspirant active ingredient so as to delay release of a portion of the active ingredient. For example, the delayed release component and antiperspirant active ingredient may be chemically attracted. In one example, the at least one delayed release component is a solid up to about 33 degrees Celsius (° C.) and has a melting point of about 33° C. to 41° C. As the delayed release component melts the antiperspirant active ingredient is released to reduce perspiration. More information about the at least one delayed release component will be described in later parts of this specification.

In accordance with the examples of the present specification, the heat activated antiperspirant composition may be in the form of an anhydrous stick. In one example, the anhydrous stick includes a number of ingredients. The anhydrous stick includes the antiperspirant active ingredient to reduce perspiration. The antiperspirant active ingredient may include astringent metallic salts, especially inorganic and organic salts of aluminum, zirconium, and zinc, as well as mixtures thereof. Particularly preferred are aluminum-containing and/or zirconium-containing salts or materials, such as aluminum halides, aluminum chlorohydrates, aluminum hydroxyhalides, zirconyl oxyhalides, zirconyl hydroxyhalides, and mixtures thereof. Exemplary aluminum salts include those having the general formula Al₂(OH)ₐCl_{b}x(H₂O), wherein a is from 2 to about 5; the sum of a and b is about 6; x is from about 1 to about 6; and wherein a, b, and x may have non-integer values. Exemplary zirconium salts include those having the general formula ZrO(OH)₂₋ₐClₐx(H₂O), wherein a is from about 1.5 to about 1.87, x is from about 1 to about 7, and wherein a and x may both have non-integer values. Particularly preferred zirconium salts are those complexes that additionally contain aluminum and glycine, commonly known as ZAG complexes. These ZAG complexes contain aluminum chlorohydroxide and zironyl hyroxy chloride conforming to the above-described formulas. Examples of active antiperspirant compounds suitable for use in the various embodiments contemplated herein include aluminum dichlorohydrate, aluminum-zirconium octachlorohydrate, aluminum sesquichlorohydrate, aluminum chlorohydrex propylene glycol complex, aluminum dichlorohydrex propylene glycol complex, aluminum sesquichlorohydrex propylene glycol complex, aluminum chlorohydrex polyethylene glycol complex, aluminum dichlorohydrex polyethylene glycol complex, aluminum sesquichlorohydrex polyethylene glycol complex, aluminum-zirconium trichlorohydrate, aluminum zirconium tetrachlorohydrate, aluminum zirconium pentachlorohydrate, aluminum zirconium octachlorohydrate, aluminum zirconium trichlorohydrex glycine complex, aluminum zirconium tetrachlorohydrex glycine complex, aluminum zirconium pentachlorohydrex glycine complex, aluminum zirconium octachlorohydrex glycine complex, zirconium chlorohydrate, aluminum chloride, aluminum sulfate buffered, and the like, and mixtures thereof. In an example, the antiperspirant active ingredient is aluminum zirconium pentachlorohydrex. In another example, the antiperspirant active ingredient is an active antiperspirant compound present in the amount of 0 to about 25 weight percent. As used herein, weight percent of an antiperspirant salt is calculated as anhydrous weight percent in accordance with the United States Pharmacopeiamethod, as is known in the art. This calculation excludes any bound water and glycerin. In yet another example, the antiperspirant active ingredient includes aluminum zirconium pentachlorohydrex at an active level of about 18 wt % to about 24 wt %.

In one example, the antiperspirant active ingredient may be an aluminum sesquichlorohydrate, aluminum dichlorohydrate, aluminum chlorohydrex propylene glycol (PG) or aluminum chlorohydrex polyethylene glycol (PEG), aluminum or aluminum zirconium glycol complexes, e.g. aluminum or aluminum zirconium propylene glycol complexes, aluminum sesquichlorohydrex PG or aluminum sesquichlorohydrex PEG, aluminum PG dichlorohydrex or aluminum PEG dichlorohydrex, aluminum hydroxide, also selected from the aluminum zirconium chlorohydrates, such as aluminum zirconium trichlorohydrate, aluminum zirconium tetrachlorohydrate, aluminum zirconium pentachlorohydrate, aluminum zirconium octachlorohydrate, the aluminum zirconium chlorohydrate glycine complexes, such as aluminum zirconium trichlorohydrex glycine, aluminum zirconium tetrachlorohydrex glycine, aluminum zirconium pentachlorohydrex glycine, aluminum zirconium octachiorohydrex glycine, potassium aluminum sulfate (KAl(SO₄)₂.12H₂O, alum), dehydrated alum (KAl(SO₄)₂ with zero to 11 moles of water of crystallization), aluminum undecylenoyl collagen amino acid, sodium aluminum lactate+aluminum sulfate, sodium aluminum chlorohydroxylactate, aluminum bromohydrate, aluminum chloride, the aluminum salts of lipoamino acids, aluminum sulfate, aluminum lactate, aluminum chlorohydroxyallantoinate, sodium aluminum chlorohydroxylactate, zinc chloride, zinc sulfocarbolate, zinc sulfate, zirconyl oxyhalides, in particular zirconyl oxychlorides, zirconyl hydroxyhalides, in particular zirconyl hydroxychlorides (zirconium chlorohydrate). Further, the antiperspirant active ingredient may form about 8 wt % to about 25 wt % of the heat activated antiperspirant composition.

In an example, the anhydrous stick further includes a dispersant such as cyclomethicone to disperse the antiperspirant active ingredient. In one example, the dispersant may be cyclopentasiloxane and/or cyclotetrasiloxane. The dispersant may form about 8 wt % to about 30 wt % of the heat activated antiperspirant composition.

Further, in an example, the anhydrous stick includes a first structurant such as a fatty alcohol. In one example, the first structurant may be stearyl alcohol. The first structurant may form about 10 wt % to about 40 wt % of the heat activated antiperspirant composition.

In an example, the anhydrous stick includes a residue masking emollient such as an alkoylated alcohol. In one example, the alkoylated alcohol may be PPG-14 butyl ether. Further, the alkoylated alcohol may form between 0 to about 20 wt % of the heat activated antiperspirant composition.

In an example, the anhydrous stick further includes a second structurant such as a second fatty alcohol. In one example, the second structurant may be cetyl alcohol. Further, the second structurant may form between 0 to 10 wt % of the heat activated antiperspirant composition.

Further, in an example, the anhydrous stick includes structurant thickeners such as fats and oils. In one example, the fats and oils may include hydrogenated castor oil. Further, the fats and oils may form between 0 to 10 wt % of the heat activated antiperspirant composition.

In an example, the anhydrous stick includes an emollient such as an ester. In one example, the ester may be myristyl myristate. Further, the ester may form between 0 to about 10 wt % of the heat activated antiperspirant composition.

In an example, the anhydrous stick includes a structurant or thickener such as synthetic polymer. In one example, the synthetic polymer may be polyethylene. Further, the synthetic polymer may form between 0 to 5 wt % of the heat activated antiperspirant composition.

Further, in an example, the anhydrous stick includes a slip modifier or bulking agent such as an inorganic mineral or oxide. In one example, the inorganic mineral or oxide may be talc. Further, the inorganic mineral or oxide may form between 0 to 10 percent of the heat activated antiperspirant composition.

In an example, the anhydrous stick includes a heat releasing material for the antiperspirant active ingredient such as the at least one delayed release component. In one embodiment, the delayed release component can be a polyethylene glycol having a molecular weight (weight average) of about 700 to about 1400. In another embodiment, the polyethylene glycol can have a weight average molecular weight of 800 to 1250, or 950 to 1050. Blends of polyethylene glycols that provide the requisite melting point properties can also be used. In one example, the at least one delayed release component may be polyethylene glycol 1000 (PEG 1000) available under the trade name Carbowax 1000 Sentry grade manufactured by the DOW chemical company. Further, the at least one delayed release component may form about 0.1 wt % to 5 wt % of the heat activated antiperspirant composition. As will be described in other parts of this specification, the at least one delayed release component is a solid at 33° C. and has a melting point about 33 to 41° C. Further, the at least delayed release component has a water solubility of at least 50 percent. In other examples, the at least one delayed release component is polyethylene glycol 300 (PEG 300) combined with polyethylene glycol 1450 (PEG 1450) which is available under the trade name Carbowax 540 Blend manufactured by the DOW chemical company.

Further, in an example, the anhydrous stick includes a volatile emollient such as cyclomethicone. In one example, the cyclomethicone may be cyclopentasiloxane. Further, the volatile emollient may form between 0 to about 10 wt % of the heat activated antiperspirant composition.

In an example, the anhydrous stick includes a perfume to give the heat activated antiperspirant composition an aesthetically pleasing smell such as a first fragrance. Further, the first fragrance may form between 0 to about 3 wt % of the heat activated antiperspirant composition.

Further, in an example the anhydrous stick includes a second fragrance for odor masking purposes. Further, the second fragrance may form between 0 to about 2 wt % of the heat activated antiperspirant composition.

In an example, the anhydrous stick includes an encapsulated fragrance such as a second perfume to give the heat activated antiperspirant composition an aesthetically pleasing smell. Further, the encapsulated fragrance may form between 0 to about 1 wt % of the heat activated antiperspirant composition.

Further, in an example, the anhydrous stick includes a starch or a modified starch for time release of the fragrances. In one example, the starch or a modified starch may be zea mays corn starch. Further, the starch or a modified starch may form between 0 to about 1 wt % of the heat activated antiperspirant composition.

An overall process for manufacturing the anhydrous stick will now be described. In one example, the process for manufacturing the anhydrous stick includes adding a heat-tolerant liquid emollient into a tank. Further, the process for manufacturing the anhydrous stick includes adding a primary structurant such as stearyl alcohol to the tank, mixing, and adding heat. The process for manufacturing the anhydrous stick further includes adding secondary structurants and non-volatile emollients and melting these ingredients with the ingredients already in the tank. Further, the process for manufacturing the anhydrous stick includes adding a structurant or thickener such as polyethylene and heating past its melting point with mixing. The batch temperature is dropped to just above batch melting point. The process for manufacturing the anhydrous stick includes mixing an active premix with a liquid dispersant until homogenous and adding the homogenous active premix to the tank keeping the ingredients in the tank above a batch melting point. Further, the process for manufacturing the anhydrous stick may include adding a slip modifier or bulking agent such as talc to the tank and mixing vigorously until homogenous. The process for manufacturing the anhydrous stick further includes adding the at least one delayed release component that is a solid up to about 33° C. and has a melting point of about 33 to 41° C., such as PEG 1000 and mixing until it is melted and droplets are dispersed throughout the anhydrous ingredients in the tank. Further, the process for manufacturing the anhydrous stick includes cooling the mixture to just above batch melting point and adding fragrances. The process for manufacturing the anhydrous stick includes cooling the ingredients in the tank to the minimum temperature to remain liquid and pouring the ingredients in the tank into canisters, snapping in twist up bases to the canisters, and cooling the ingredients in the canisters before inverting the anhydrous sticks.

In accordance with the examples of the present specification, the heat activated antiperspirant composition may be a gel. In an example the gel includes an oil phase and a water phase. The oil phase may include cyclomethicone, dimethicone copolyol, trisiloxane, dimethicones, and one or more fragrances that include an odor masking fragrance, an odor inhibiting fragrance, a fragrance, or combinations thereof. Further, the oil phase may form about 10 wt % to about 25 wt % of the heat activated antiperspirant composition. The water phase may include water as a diluent, an antiperspirant active ingredient, water as an active solvent, calcium chloride, propylene glycol or dipropylene glycol, ethanol, the at least one delayed release component that is a solid up to about 33° C. and has a melting point of about 33° C. to about 41° C., or combinations thereof. Further, the water phase may form about 75 wt % to about 90 wt % of the heat activated antiperspirant composition.

The gel may include a diluent such as cyclomethicone mixed with a copolymer. In one example, a diluent may be a diluting agent. In an example, specific fluids may be too viscous to be pumped easily or too dense to flow from one particular point to the other. To ease this restricted movement, diluents are added. This decreases the viscosity of the fluids. In an example, the copolymer and cyclomethicone mixture may be 85 wt % cyclopentasiloxane, 10 wt % PEG/PEG-18/18, and 5 wt % water. Further, the cyclomethicone may form about 3 wt % to about 12 wt % of the heat activated antiperspirant composition.

In an example, the gel may include an emulsifier copolymer such as dimethicone copolyol. Emulsifiers are substances that can mix ingredients that otherwise don't mix. To achieve this, emulsifiers have a hydrophobic end that is attracted to the oil molecules and a hydrophilic end that is attracted to the water molecules, consequently bringing the two substances together. In one example, the dimethicone copolyol may be in a mixture with cyclomethicone. Further, the dimethicone copolyol may form about 0.5 wt % to about 3 wt % of the heat activated antiperspirant composition.

Further, in an example, the gel can include an emollient for skin feel such as the trisiloxane. In one example, the trisiloxane may be in a mixture with dimethicones. For example, the mixture may be about 75 percent trisiloxane and about 25 percent dimethicones. Further, the trisiloxane may form between 0 to about 4 wt % of the heat activated antiperspirant composition.

As mentioned above, the gel includes an emollient for skin feel such as the dimethicones. In one example, the dimethicones may be polydimethylsiloxane or decamethyltetrasiloxane or dodecamethylpentasiloxane. Further, the dimethicones may form about 2 wt % to about 10 wt % of the heat activated antiperspirant composition.

In an example, the gel includes the odor masking fragrance to mask odors. The odor masking fragrance may form between 0 to about 1 wt % of the heat activated antiperspirant composition.

As mentioned above, the gel includes the odor inhibiting fragrance to inhibit odors. The odor inhibiting fragrance may form between 0 to about 1 wt % of the heat activated antiperspirant composition.

Further, in an example, the gel includes the fragrance such as a perfume to give the heat activated antiperspirant composition an aesthetically pleasing smell. The fragrance may form 0 to about 2.00 wt % of the heat activated antiperspirant composition.

As mentioned above, the gel includes a diluent such as water. The diluent may form between 0 to about 15 wt % of the heat activated antiperspirant composition.

In an example, the gel includes the antiperspirant active ingredient to reduce perspiration. The antiperspirant active ingredient may be aluminum or aluminum/zirconium. Further, the antiperspirant active ingredient may form about 8 to about 25 wt % of the heat activated antiperspirant composition.

As mentioned above, the gel includes an active solvent such as the water. The active solvent may form about 8 wt % to 30 wt % of the heat activated antiperspirant composition.

Further, in an example, the gel includes an active stabilizer or activator such as the calcium chloride. The calcium chloride may form about 0 to about 5 wt % of the heat activated antiperspirant composition.

As mentioned above, the gel may include an emollient or humectant such as propylene glycol or dipropylene glycol. In one example, the humectant is a hygroscopic substance used to keep the gel moist. Further, the propylene glycol or dipropylene glycol may form between 0 to about 15 wt % of the heat activated antiperspirant composition.

In an example, the gel includes a volatile diluent or antibacterial such as ethanol or isopropanol. The ethanol or isopropanol may form about 5 wt % to about 15 wt % of the heat activated antiperspirant composition.

As mentioned above, the gel includes the at least one delayed release component which is a solid up to about 33° C. and has a melting point about 33° C. to 41° C. and is a heat releasing material for the antiperspirant active ingredient. In one example, the at least one delayed release component may be PEG 1000. Further, the at least one delayed release component may form about 0.02 to about 2 percent of the heat activated antiperspirant composition. In other examples, the at least one delayed release component may be a mixture of PEG 300 and PEG 1450 and is available under the trade name Carbowax 540 blend manufactured by the DOW Chemical Company. Further, the at least one delayed release component has a water solubility of at least 50 percent.

An overall process for manufacturing the gel will now be described. In one example, the process for manufacturing the gel includes mixing the oil phase ingredients until uniformity is obtained in a first tank. The process for manufacturing the gel includes mixing the water phase ingredients until uniformity is obtained in a second tank. Further, the water phase is slowly added to the oil phase with adequate mixing to build a low viscosity water-in-oil emulsion. The low viscosity emulsion is mixed with high shear until viscosity reaches about 100,000 centipoise. In one example, gel antiperspirants may match a refractive index of the oil phase and water phase so the gel is clear.

In accordance with the examples of the present specification, the heat activated antiperspirant composition may be an emulsion stick. In an example, the emulsion stick includes a water phase and an oil phase. The water phase may include water as a diluent, the antiperspirant active ingredient, the at least one delayed release component, propylene glycol, or combinations thereof. The oil phase may include cyclomethicone, an ester, at least one dimethicone copolymer, at least one synthetic polymer, synthetic wax, a fragrance, or combinations thereof.

In an example, the emulsion stick includes a first diluent such as the water. In one example, the first diluent may form between 0 to about 5 wt % of the heat activated antiperspirant composition.

Further, in an example, the emulsion stick includes the antiperspirant active ingredient to reduce perspiration. In an example, the antiperspirant active ingredient may be aluminum zirconium tetracholorohydrex GLY. Further, the antiperspirant active ingredient may form about 8 wt % to about 25 wt % of the heat activated antiperspirant composition.

In an example, the water phase of the emulsion stick includes an active solvent such as water present in the aluminum zirconium tetracholorohydrex GLY. Further, the active solvent may form about 8 wt % to about 30 wt % of the heat activated antiperspirant composition.

Further, in an example, the water phase of the emulsion stick includes the at least one delayed release component which is a solid up to about 33° C. and has a melting point of about 33 to about 41° C. and is a heat releasing material for the antiperspirant active ingredient. In one example, the at least delayed release component may be PEG 1000. Further, the at least one delayed release component may form of about 0.1 wt % to about 5 wt % of the heat activated antiperspirant composition. In other examples, the at least one delayed release component may be a mixture of PEG 300 and PEG 1450 and has a melting point of about 33° C. to about 41° C. and is available under the trade name Carbowax 540 Blend manufactured by the DOW Chemical Company. Further, the at least one delayed release component has a water solubility of at least 50 wt %.

In an example, the water phase of the emulsion stick includes a humectant such as the propylene glycol. Further, the propylene glycol may form between 0 to about 5 wt % of the heat activated antiperspirant composition.

Further, in an example, the oil phase of the emulsion stick includes an emollient to enhance skin feel such as the cyclomethicone. Emollients are complex mixtures of chemical agents to make the external layers of the skin softer and more pliable. Further, the cyclomethicone may form about 5 wt % to about 15 wt % of the heat activated antiperspirant composition.

In an example, the oil phase of the emulsion stick includes an emollient such as the ester. In one example, the ester may be C12-15 Alkyl Benzoate. Further, the ester may form between 0 to about 12 wt % of the heat activated antiperspirant composition.

Further, in an example, the oil phase of the emulsion stick includes a first skin conditioner or emulsifier such as the dimethicone copolymer. In an example, the first skin conditioner or emulsifier may be Cetyl PEG/PPG-10/1Dimethicone. Further, the first skin conditioner or emulsifier may form about 0.2 to about 3 wt % of the heat activated antiperspirant composition.

In an example, the oil phase of the emulsion stick includes a second skin conditioner or emulsifier such as the dimethicone copolymer. In an example, the second skin conditioner or emulsifier may be Bis—PEG/PPG—14/14 Dimethicone/Dimethicone. Further, the second skin conditioner or emulsifier may form between 0 to about 3 wt % of the heat activated antiperspirant composition.

Further, in an example the oil phase of the emulsion stick includes an emulsion stabilizer or structurant such a higher melting point synthetic polymer. In an example, the synthetic polymer may be polyethylene which has a melting point of about 75° C. to about 90° C. Further, the higher melting point synthetic polymer may form between 0 to about 2 wt % of the heat activated antiperspirant composition.

In an example, the oil phase of the emulsion stick includes an emulsion stabilizer or structurant such as a lower melting point synthetic polymer. In an example, the synthetic polymer may be polyethylene which has a melting point of about 65° C. to about 75° C. Further, the lower melting point synthetic polymer may form about 2 wt % to about 14 wt % of the heat activated antiperspirant composition.

Further, in an example, the oil phase of the emulsion stick includes an emulsion stabilizer or structurant such as a synthetic wax. The synthetic wax may form between 0 to about 5 wt % of the heat activated antiperspirant composition.

In an example, the oil phase of the emulsion stick includes the fragrance such as a perfume to give the heat activated antiperspirant composition an aesthetically pleasing smell. The fragrance may form between 0 to about 3 wt % of the heat activated antiperspirant composition.

An overall process for manufacturing the emulsion stick will now be described. In one example, the process for manufacturing the emulsion stick includes combining and heating the ingredients for the oil phase to a temperature above the highest melting point of the ingredients in a first tank. The process further includes combining and heating the ingredients for the water phase to the same temperature in a second tank. Further, the process includes adding the water phase slowly to the oil phase with moderate mixing. The process further includes mixing the water phase and the oil phase for a minimum of 10 minutes at a combined batch temperature. Further, the process includes adding a fragrance to the water phase and the oil phase and mixing for 1 minute. The process further includes homogenizing the water phase and the oil phase until a fine water-in-oil emulsion is formed. Further, the process includes pouring the fine water-in-oil emulsion into packaging and let cool until hard.

As mentioned above, the heat activated antiperspirant composition includes at least one delayed release component that is a solid up to about 33° C. and has a melting point of about 33° C. to 41° C. to release an antiperspirant active ingredient to reduce perspiration. In one example, the at least one delayed release component is PEG 1000. In another example the at least delayed release component is a mixture of PEG 300 and PEG 1450 which is available under the trade name Carbowax 540 manufactured by the DOW Chemical Company, or combinations thereof. Further, the at least one delayed release component is a solid at normal skin temperature. In an example, normal skin temperature may be 33° C. which is several degrees below body core temperature. Further, the at least one delayed release component has a melting point at slightly higher than normal skin temperatures such as 33 to 41° C.

In one example, the at least one delayed release component has a melting point of about 33° C. to about 41° C. In an example, the at least one delayed release component may be PEG 1000. In another example, the at least one delayed release component has a melting point of about 34 to 38° C. In an example, the at least one delayed release component may be a mixture of PEG 300 and PEG 1450 which is available under the trade name Carbowax 540 Blend manufactured by the DOW Chemical Company. Further, the at least one delayed release component forms about 0.02 wt % to about 5 wt % of the heat activated antiperspirant composition. The at least one delayed release component for the anhydrous stick may form about 0.1 wt % to about 5 wt % of the heat activated antiperspirant composition to aid in creating holes in a matrix as the at least one delayed release component melts and dissolves into sweat.

In one example, the at least one delayed release component attracts, co-mingles, partitions, or otherwise interacts with at least a portion of the antiperspirant active ingredient so as to delay release of the antiperspirant active ingredient from the antiperspirant composition. The “delayed release” of the antiperspirant active ingredient from the antiperspirant composition generally refers to delaying the availability of the antiperspirant active ingredient to be solubilized or dissolve into eccrine sweat and to diffuse into the eccrine gland ducts. The at least one delayed release component melts when skin temperature is elevated making the antiperspirant active ingredient more available to dissolve into eccrine sweat and diffuse into the eccrine gland ducts to form a gelatinous insoluble polymeric aluminum hydroxide protein gel plug and partially plug sweat glands. For example, the incorporation of the at least one delayed release component into an anhydrous stick in the molten phase creates dispersed droplets of the at least one delayed release component which attracts the antiperspirant active ingredient. When the at least one delayed release component melts at a temperature just above skin temperature, it makes the antiperspirant ingredient more available to dissolve into sweat and diffuse into sweat glands. As the at least one delayed release component melts and also dissolves into sweat, it creates voids in the solid stick matrix of the anhydrous stick. This results in an increased surface area for perspiration to extract even more of the antiperspirant active ingredient. In one example, it is not obvious to use the at least one delayed release component in an anhydrous stick as all of the ingredients in current anhydrous sticks, besides the active, are not water soluble and form a uniform mass with no dispersed droplets.

In another example, the at least one delayed release component is incorporated into a gel by dissolving it in the water phase along with the antiperspirant active ingredient. This allows the at least one delayed release component to interact with the solubilized antiperspirant active ingredient. As a result, the at least one delayed release component is a liquid in the formulation but goes back to a solid phase after application of the heat activated antiperspirant composition when the water and/or alcohol ingredients evaporates. As mentioned above, the at least one delayed release component melts when skin temperature is elevated making the antiperspirant active ingredient more available to be dissolved into perspiration also caused by the elevated temperature and diffuse into the eccrine gland ducts to form aluminum hydroxide protein plugs. In an example, it is not obvious to use the at least one delayed release component, solid at 33° C., in gels as the current water phases contain only ingredients that are liquid at room temperature and skin temperature. The exception is the antiperspirant active ingredient which is a solid dissolved in water.

In yet another example, the at least one delayed release component is incorporated into an emulsion. In this example, the at least delayed release component, solid up to about 33° C., is dissolved in a water phase which is entrapped in the anhydrous, non-water soluble solid stick matrix. However, the water phase behaves like the gel, where the at least one delayed release component is dissolved and comingled with the active ingredient. However, the at least one delayed release component returns to being a solid at normal skin temperature, yet melts when skin temperature is elevated making the active ingredient more available to be dissolved into perspiration and diffuse into the eccrine gland ducts to form aluminum hydroxide protein plugs.

The heat activated antiperspirant composition may include carriers to facilitate a deposition of the antiperspirant activate ingredient. In this example, the carrier may be used to hold the antiperspirant active ingredient and the at least one delayed release component. Further, the at least one delayed release component is a solid up to about 33° C. and has a melting point of about 33° C. to about 41° C. to release the antiperspirant active ingredient to create vacancies in the carrier. The carrier can include one or more compositions known in the art for creating antiperspirant compositions. The vacancies allow perspiration to more readily access and dissolve the antiperspirant active ingredient As a result, the at least one delayed release component may be used to improve the availability of the antiperspirant active ingredient when the skin temperature rises.

In some examples, the heat activated antiperspirant composition is included in a heat activated antiperspirant product, in which the heat activated antiperspirant composition is included in a container. In one example, the container may be a canister. The container may be equipped with an opening capable of dispensing the heat activated antiperspirant composition. The opening may dispense the heat activated antiperspirant composition by a twist up base type mechanism, or another appropriate dispenser. The opening dispenses the heat activated antiperspirant composition onto an area of the skin. For example, the opening may dispense the heat activated antiperspirant composition onto an underarm of a user. Further, the container may have a cap to cap off the heat activated antiperspirant composition.

Table (1) gives an example of the heat activated antiperspirant composition in the form of an anhydrous stick as described in the present specification. More specifically, the heat activated antiperspirant composition of Table (1) includes the below listed ingredients in the indicated weight percentages.

TABLE (1)

| | Weight |
|---|---|
| | Percent |
| Ingredient | (%) |
| Antiperspirant Active Ingredient | 8-25 |
| Dispersant (Cyclomethicone) | 8-30 |
| First Structurant (Fatty Alcohol) | 10-40 |
| Alkoylated Alcohol | 0-20 |
| Second Structurant (Fatty Alcohol) | 0-10 |
| Fats and Oils | 0-10 |
| Ester | 0-10 |
| Synthetic Polymer | 0-5 |
| Inorganic Mineral or Oxide | 0-10 |
| Delayed Release Component (Melting Point 34-41° C.) | 0.1-5 |
| Volatile Emollient (Cyclomethicone) | 0-10 |
| First Fragrance | 0-3 |
| Second odor masking Fragrance | 0-2 |
| Encapsulated Fragrance | 0-1 |
| Starch or Modified Starch | 0-1 |

As illustrated in Table (1), the cyclomethicone may be used to disperse the antiperspirant active ingredient and used as a volatile emollient. Further, the fatty alcohols may be stearyl alcohol and cetyl alcohol.

Table (2) gives an example of the heat activated antiperspirant composition in the form of an emulsion stick as described in the present specification. More specifically, the heat activated antiperspirant composition of Table (2) includes the below listed ingredients in the indicated weight percentages.

TABLE (2)

| | Weight |
|---|---|
| | Percent |
| Ingredient | (%) |
| First Diluent (Water) | 0-5 |
| Antiperspirant Active Ingredient | 8-25 |
| Active Solvent (Water) | 8-30 |
| Delayed Release Component (Melting Point 34-41° C.) | 0.02-2 |
| Propylene Glycol | 0-5 |
| Cyclomethicone | 5-15 |
| Ester | 0-12 |
| First Skin Conditioner or Emulsifier | 0.2-3 |
| (Dimethicone Copolymer) | |
| Second Skin Conditioner or Emulsifier | 0-3 |
| (Dimethicone Copolymer) | |
| High Melting Point Synthetic Polymer | 0-2 |
| Low Melting Point Synthetic Polymer | 2-14 |
| Synthetic Wax | 0-5 |
| Fragrance | 0-3 |

As illustrated in Table (2), one water element may be a diluent and the other water may be a specific diluent of an antiperspirant active ingredient. The dimethicone copolymers may be cetyl PEG/PPG-10/1 dimethicone and Bis-PEG/PPG-14/14 dimethicone/dimethicone. The synthetic polymers may be polyethylene with a melting point of about 65° C. to 90° C.

Table (3) gives an example of the heat activated antiperspirant composition in the form of a gel as described in the present specification. More specifically, the heat activated antiperspirant composition of Table (3) includes the below listed ingredients in the indicated weight percentages.

TABLE (3)

| | Weight |
|---|---|
| | Percent |
| Ingredient | (%) |
| Oil Phase | 10-25 |
| Cyclomethicone | 3-12 |
| Dimethicone Copolyol | 0.5-3 |
| Trisiloxane | 0-4 |
| Dimethicone | 2-10 |
| Odor Masking Fragrance | 0-1 |
| Odor Inhibiting Fragrance | 0-1 |
| Fragrance | 0-2 |
| Water Phase | 75-90 |
| Diluent (Water) | 0-15 |
| Active Ingredient | 8-25 |
| Active Solvent (Water) | 10-30 |
| Calcium Chloride | 0-5 |
| Propylene glycol or Dipropylene Glycol | 0-15 |
| Ethanol or isopropanol | 5-15 |
| Delayed Release Component (Melting Point 34-41° C.) | 0.02-2 |

As demonstrated above, the heat activated antiperspirant composition is unique in that the heat activated antiperspirant composition includes at least one delayed release component that is a solid up to about 33° C. and has a melting point of about 33° C. to about 41° C. to release an antiperspirant active ingredient to reduce perspiration. As a result, the at least one delayed release component melts when skin temperature is elevated making the antiperspirant active ingredient more available to be dissolved into perspiration also caused by the elevated temperature which dissolves the antiperspirant active ingredient and diffuses into the eccrine gland ducts to form a gelatinous insoluble polymeric aluminum hydroxide protein gel plug and partially plug sweat glands to reduce perspiration.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A heat activated antiperspirant composition, comprising:
an antiperspirant active ingredient; and
at least one delayed release component having a melting point of about 33° C. to 41° C.

2. The heat activated antiperspirant composition of claim 1, wherein the delayed release component delays the release of a portion of the antiperspirant active ingredient.

3. The heat activated antiperspirant composition of claim 1, wherein the heat activated antiperspirant composition is in the form of an anhydrous stick, the heat activated antiperspirant composition further comprising at least one of a dispersant, an alkoxylated alcohol, or combinations thereof.

4. The heat activated antiperspirant composition of claim 1, wherein the heat activated composition is in the form of a gel, the heat activated antiperspirant composition further comprising at least one of cyclomethicone, dimethicone copolyol, trisiloxane, dimethicones, calcium chloride, propylene glycol, ethanol, or combinations thereof.

5. The heat activated antiperspirant composition of claim 1, wherein the heat activated composition is in the form of an emulsion stick, the heat activated antiperspirant composition further comprising a first diluent, propylene glycol, cyclomethicone, a skin conditioner, a high melting point synthetic polymer, a low melting point synthetic polymer, synthetic wax, a fragrance, or combinations thereof.

6. The heat activated antiperspirant composition of claim 1, wherein the delayed release component is a polyethylene glycol having a molecular weight of 700 to 1400.

7. The heat activated antiperspirant composition of claim 1, wherein the delayed release component includes polyethylene glycol 1000 (PEG 1000).

8. The heat activated antiperspirant composition of claim 1, wherein the delayed release component is a mixture of polyethylene glycol 300 (PEG 300) and polyethylene glycol 1450 (PEG 1450).

9. The heat activated antiperspirant composition of claim 1, wherein the delayed release component has a melting point of about 34° C. to about 38° C.

10. The heat activated antiperspirant composition of claim 1, wherein the delayed release component comprises about 0.02 wt % to about 5 wt % of the heat activated antiperspirant composition.

11. The heat activated antiperspirant composition of claim 1, further comprising a carrier.

12. The heat activated antiperspirant composition of claim 11, wherein when the antiperspirant composition is applied to a skin surface of a subject and the temperature of the skin surface rises to above about 33° C. at least a portion of the delayed release component melts and releases a portion of the antiperspirant active ingredient.

13. The heat activated antiperspirant composition of claim 12, wherein the melting of the delayed release component forms pores in the applied antiperspirant composition increasing the surface area of the applied antiperspirant composition.

14. A heat activated antiperspirant product, comprising:
a container; and
a heat activated antiperspirant composition disposed within the container, the heat activated antiperspirant composition comprising;
an antiperspirant active ingredient; and
at least one delayed release component, the delayed release component having a melting point of about 33° C. to about 41° C.

15. The heat activated antiperspirant product of claim 14, wherein the delayed release component is polyethylene glycol 1000 (PEG 1000).

16. The heat activated antiperspirant product of claim 14, wherein the delayed release component is a mixture of polyethylene glycol 300 (PEG 300) and polyethylene glycol 1450 (PEG 1450).

17. The heat activated antiperspirant product of claim 14, wherein the delayed release component has a melting point of about 34° C. to about 38° C.

18. The heat activated antiperspirant product of claim 14, wherein the delayed release component comprises about 0.02 wt % to 5 wt % of the heat activated antiperspirant composition.
